(19) United States
(12) Patent Application Publication
(10) Pub. No.: US 2021/0329992 A1
(43) Pub. Date: Oct. 28, 2021

(54) FACE SHIELD FOR USE WITH A PHOROPTER

(71) Applicant: VisibleBliss, LLC, Argyle, TX (US)

(72) Inventor: Elvin Fenton, Argyle, TX (US)

(21) Appl. No.: 17/242,076

(22) Filed: Apr. 27, 2021

Related U.S. Application Data
(60) Provisional application No. 63/016,114, filed on Apr. 27, 2020.
(60) Provisional application No. 63/046,437, filed on Jun. 30, 2020.
(60) Provisional application No. 63/125,234, filed on Dec. 14, 2020.

(51) Int. Cl. A41D 13/11

(52) U.S. Cl. CPC A41D 13/1138

(57) ABSTRACT

A face shield for use with phoropters is described with methods for using the same. The face shield includes a central cavity, which is defined by a front wall and one or more sidewalls extending outwardly from a perimeter of the front wall. The central cavity is configured for surrounding a nasal region, an oral region, and a mental region of an adult person's face. The face shield includes one or more attachment features extending outwardly from the central cavity and configured for attaching the face shield to the phoropter. A method for securing a face shield including attachment features and a central cavity defining an upper cavity region, a middle cavity region, and a lower cavity region to a phoropter is described. The method includes positioning the upper cavity region between lens housings of the phoropter and attaching the attachment features to securing components of the phoropter.

### CROSS-REFERENCE TO RELATED APPLICATIONS

This application claims the benefit of U.S. Provisional Patent Application Ser. No. 63/016,114 filed on Apr. 27, 2020, which is incorporated herein by reference in its entirety, including any figures, tables, and drawings. This application also claims the benefit of U.S. Provisional Patent Application Ser. No. 63/046,437 filed on Jun. 30, 2020, which is incorporated herein by reference in its entirety, including any figures, tables, and drawings. This application also claims the benefit of U.S. Provisional Patent Application Ser. No. 63/125,234 filed on Dec. 14, 2020, which is incorporated herein by reference in its entirety, including any figures, tables, and drawings.

### FIELD OF THE INVENTION

The embodiments described herein relate generally to personal protective equipment and, in particular, to face shields configured for use with phoropters.

### BACKGROUND

In order to determine the refractive properties of an eye for the purpose of providing corrective lenses, optometrists commonly use a phoropter. The patient sits behind the phoropter and views a diagram. Optical elements of different spherical power and different cylindrical correction are inserted in front of the patient's eye or eyes. The patient tells the optometrist which lens provides the best vision. This process is repeated with multiples lenses until the best lens or combination of lenses is found for the patient.

Testing with a phoropter requires close proximity of the optometrist and the patient. To protect one another from possible transmission of infectious disease, both the patient and the optometrist can wear a facial covering. However, medical masks are not often used by both the optometrist and the patient, and nonmedical masks may not be considered sufficiently effective at blocking transmission of infectious disease. Further, the use of a more effective mask, such as a respirator, will not allow the patient to position properly against the phoropter.

More recently, the outbreak of Coronavirus disease 2019 (COVID-19) has resulted in an increased risk of viral transmission between optometrists and patients. This is especially true due to the proximity of the optometrist's face to the patient's face during optical exams. Thus, there is a need in the art for personal protective equipment that can be used with a phoropter to reduce the transmission of infectious disease carried by viral and bacterial emissions from the nose and throat.

### BRIEF SUMMARY

Various embodiments described herein are directed to a face shield configured for use with a phoropter. In some embodiments, the face shield defines a central cavity to accommodate the nasal and oral region of a person. In some embodiments, the face shield can be attached to the phoropter with pre-existing cheek guard clips on the phoropter. In other embodiments, in the absence of cheek guard clips on the phoropter, the face shield includes a loop or halter to be secured around a knob or forehead rest of the phoropter. In some embodiments, the face shield is made of a thin, flexible, disposable plastic sheet.

In accordance with one aspect, a face shield for use with a phoropter is provided. The face shield includes a central cavity defined by a front wall and one or more sidewalls extending outwardly from a perimeter of the front wall. The central cavity is formed from an impermeable material. The central cavity is configured for surrounding a nasal region, an oral region, and a mental region of an adult person's face. The face shield further includes one or more attachment features extending outwardly from the central cavity and configured for attaching the face shield to the phoropter.

In an example embodiment, the sidewalls of the face shield include two lateral sidewalls, a bottom sidewall, and a top sidewall. In an example embodiment, the central cavity of the face shield defines a plurality of cavity regions. The plurality of cavity regions includes an upper cavity region, a middle cavity region, and a lower cavity region. The upper region of the central cavity defines a width configured to accommodate the nasal region of the adult person's face. The middle region of the central cavity defines a width configured to accommodate the oral region of the adult person's face. The lower region of the central cavity defines a width configured to accommodate the mental region of an adult person's face. In an example embodiment, the width defined by the central cavity's upper region is less than the width defined by the central cavity's middle region and less than the width defined by the central cavity's lower region. In an example embodiment, the width of the central cavity's upper region is further configured to be positioned between lens housings of the phoropter. In an example embodiment, the central cavity defines a cavity opening. The one or more attachment features extend outwardly from the one or more sidewalls in a plane substantially parallel to a plane of the cavity opening.

In an example embodiment, the one or more attachment features of the face shield include a first side tab extending outwardly and upwardly from a first side of the central cavity. The one or more attachment features further include a second side tab extending outwardly and upwardly from a second side of the central cavity. In an example embodiment, each of the first side tab and the second side tab are configured to be secured to a surface of the phoropter by preexisting cheek guard clips. In an example embodiment, the one or more attachment features include an upper flange extending upwardly from the central cavity and defining an aperture above the central cavity. In an example embodiment, an inner perimeter of the upper flange's aperture defines a notch configured to be positioned on an outwardly protruding feature of the phoropter. In an example embodiment, the face shield is formed form a single piece of impermeable material selected from a group consisting of: polyethylene, polypropylene, polyethylene, terephthalate, and acrylics.

In accordance with another aspect, a method for securing a face shield to a patient-side of a phoropter for protection of a patient is provided. The face shield includes a central cavity defined by a front wall and one or more sidewalls, and the face shield's central cavity defines an upper cavity region, a middle cavity region, and a lower cavity region. The face shield further comprises one or more attachment features extending outwardly from the central cavity. The provided method includes positioning the upper cavity region of the face shield between lens housings of the phoropter. The provided method further includes attaching the one or more attachment features of the face shield to one or more securing components of the patient-side of the phoropter.

In an example embodiment, the face shield's one or more attachment features include two side tabs, and the phoropter's one or more securing components include two preexisting cheek guard clips attached to and biased towards a surface of the phoropter. The step of attaching the one or more attachment features of the face shield to one or more securing components of the phoropter then includes securing each of the face shield's side tabs between one or the preexisting cheek guard clips and the surface of the phoropter.

In an example embodiment, the face shield's one or more attachment features include an upper flange defining an aperture above the central cavity, and the phoropter's one or more securing components include an outwardly protruding member. The step of attaching the one or more attachment features of the face shield to one or more securing components of the phoropter then includes positioning the face shield's upper flange on the outwardly protruding member such that the outwardly protruding member is positioned within the upper flange's aperture.

### BRIEF DESCRIPTION OF THE DRAWINGS

The accompanying drawings, which are incorporated in and constitute a part of this specification, illustrate one or more implementations described herein and, together with the description, explain these implementations. The drawings are not intended to be drawn to scale, and certain features and certain views of the figures may be shown exaggerated, to scale, or in schematic in the interest of clarity and conciseness. Not every component may be labeled in every drawing. Any like reference numerals in the figures may represent and refer to the same or similar element or function. In the drawings:

FIG. 1 is a perspective view of a patient-side of a phoropter face shield according to one embodiment.

FIG. 2 is a perspective view of an optometrist-side of the phoropter face shield of FIG. 1.

FIG. 3 is a front view of the patient-side of the phoropter face shield of FIG. 1.

FIG. 4 is a front view of the optometrist-side of the phoropter face shield of FIG. 1.

FIG. 5 is a side view of the phoropter face shield of FIG. 1.

FIG. 6 is a top view of the phoropter face shield of FIG. 1.

FIG. 7 is a bottom view of the phoropter face shield of FIG. 1.

FIG. 8 is a front view of the patient-side of the phoropter face shield of FIG. 1 in which widths of the face shield's central cavity are indicated.

FIG. 9 is a front view of the patient-side of the phoropter face shield of FIG. 1 in which regions of the face shield's central cavity are indicated.

FIG. 10 is a perspective view of the phoropter face shield of FIG. 1 secured to a manually operated phoropter having laterally positioned clips according to one embodiment.

FIG. 11 is a perspective view of an automated phoropter having an outwardly protruding forehead rest.

FIG. 12 is a perspective view of an automated phoropter having an outwardly protruding knob.

FIG. 13 is a perspective view of a patient-side of a phoropter face shield according to another embodiment.

FIG. 14 is a perspective view of an optometrist-side of the phoropter face shield of FIG. 13 according to one embodiment.

FIG. 15 is a front view of the patient-side of the phoropter face shield of FIG. 13 according to one embodiment.

FIG. 16 is a front view of the optometrist-side of the phoropter face shield of FIG. 13 according to one embodiment.

FIG. 17 is a side view of the phoropter face shield of FIG. 13 according to one embodiment.

FIG. 18 is a top view of the phoropter face shield of FIG. 13 according to one embodiment.

FIG. 19 is a bottom view of the phoropter face shield of FIG. 13 according to one embodiment.

FIG. 20 is a perspective view of an anatomical diagram of an adult person's face.

### DETAILED DESCRIPTION

Described herein are various embodiments of a face shield configured to be attached to a phoropter (e.g., a manually operated phoropter or automated phoropter). In various embodiments, the face shield comprises an impermeable sheet (e.g., formed from molded plastic) generally defining a central cavity shaped to mask a patient's nasal, oral, and mental regions (e.g., a patient's nose and mouth). As described in greater detail herein, the face shield also includes attachment features for securing the face shield to a phoropter.

In various embodiments, the face shield is configured to be secured to a patient-side of a phoropter (i.e., the back side opposite the side facing the optometrist) in order to protect the patient and shield the phoropter, associated equipment, and optometrist from contamination. The patient-side of the phoropter is typically equipped with cheek guards where the patient is instructed to rest his or her face. The cheek guards can be wiped clean between patients. However, most phoropters, particularly manually operated phoropters, are equipped with cheek guard clips (e.g., stainless steel clips) for attaching disposable cheek guard covers over the cheek guards. These disposable cheek guard covers can be replaced with fresh cheek guard covers for each patient and thus eliminate the need to wipe clean the cheek guards between patients. As described herein, certain embodiments of the face shield are configured for being secured to the phoropter using these clips. In particular, certain embodiments of the face shield include side tabs that extend upwardly and outwardly from the face shield's lateral sides to permit the face shield to be secured by the clips. The face shield can therefore be attached to (and subsequently removed from) the phoropter without modifying the phoropter.

In another embodiment, the face shield includes an upper flange defining an aperture positioned above the central cavity and configured to permit the face shield to hang from an outwardly protruding member on the phoropter (e.g., a forehead rest or central adjusting knob). In such embodiments, the flange and aperture function as a loop or halter in order to permit the face shield to be secured to phoropters. For example, the flange and aperture permit the face shield to be secured to phoropters that may lack cheek guard clips. This embodiment also permits the face shield to be attached to (and subsequently removed from) the phoropter without modifying the phoropter.

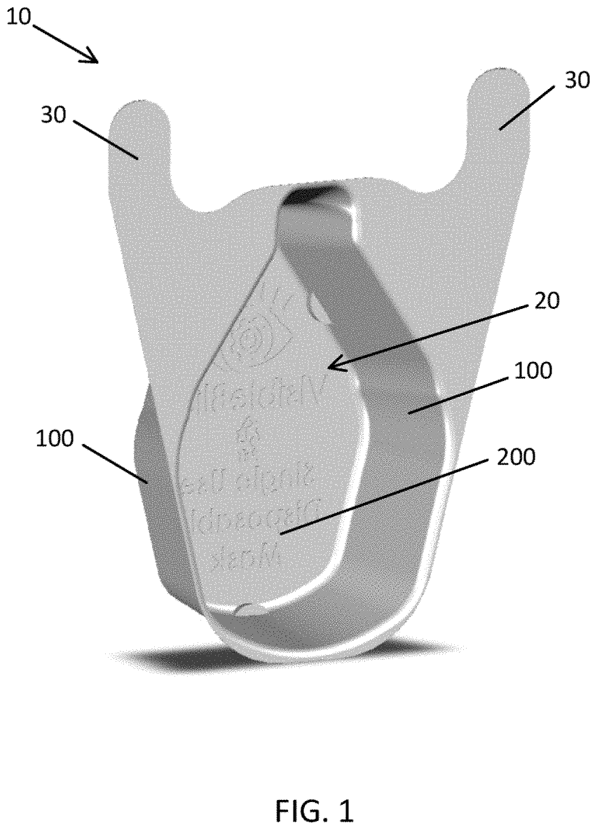

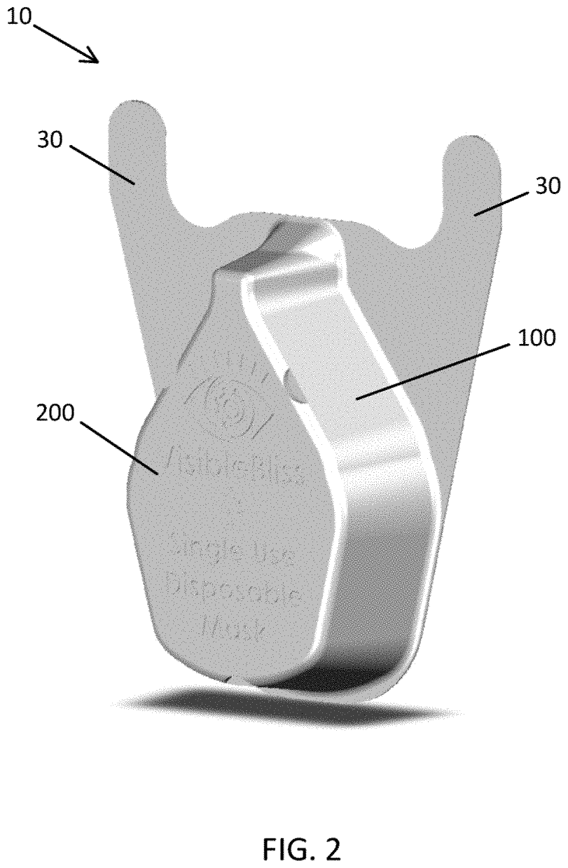

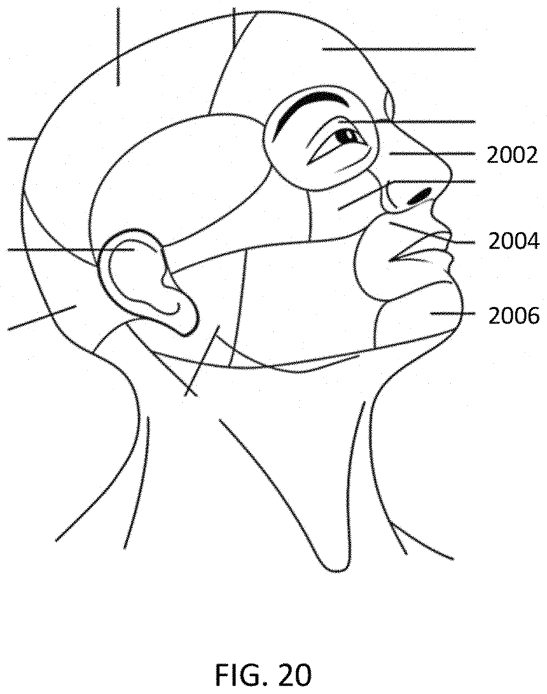

FIGS. 1 and 2 illustrate a face shield 10 according to one embodiment. As shown in the illustrated embodiment, the face shield 10 includes a central cavity 20 and side tabs 30. In various embodiments, the central cavity 20 is dimensioned for surrounding the nasal region, oral region, and mental region of a user's face. A nasal region 2002, oral region 2004, and mental region 2006 of an adult person's face is shown for context in FIG. 20. As illustrated in FIG. 20, a nasal region 2002 of an adult person's face may be a region including, surrounding, co-located with, and/or associated with the adult person's nose. An oral region 2004 of an adult person's face may be a region including, surrounding, co-located with, and/or associated with the adult person's mouth. A mental region 2006 of an adult person's face may be a region including, surrounding, co-located with, and/or associated with the adult person's chin or mental nerve.

The central cavity 20 is defined by a front wall 200 and sidewalls 100. The sidewalls 100 are positioned around the perimeter of the front wall 200 and extend outwardly from the plane defined by the front wall 200. In particular, in the illustrated embodiment, the sidewalls 100 are oriented substantially perpendicular to the front wall 200.

As shown in FIG. 1, the central cavity 20 defines a recessed area dimensioned for surrounding the nasal region 2002, oral region 2004, and mental region 2006 of a patient's face on a patient-side of the phoropter (i.e., the side of the face shield immediately adjacent to the patient's face). Likewise, as shown in FIG. 2, the central cavity's sidewalls 100 and front wall 200 protrude outwardly on the optometrist-side of the face shield 10 (i.e., the side of the face shield that is opposite the patient-side).

As further illustrated in FIGS. 1 and 2, the face shield's side tabs 30 extend upwardly and outwardly from lateral portions of the face shield 10. In particular, upper portions of the side tabs 30 extend above the central cavity 20. As discussed in greater detail herein, the side tabs 30 are configured to permit the face shield 10 to be secured to a phoropter via the phoropter's cheek guard clips. In this way, for example, the side tabs 30 are attachment features of the face shield 10.

In various embodiments, the face shield 10 is formed from a single piece of impermeable material. For example, in the illustrated embodiment of FIGS. 1 and 2, the face shield 10 is formed from a thin plastic sheet molded to define the central cavity 20 and side tabs 30. Plastics suitable for forming the face shield 10 include, but are not limited to, polyethylene, polypropylene, polyethylene terephthalate, acrylics, and the like. Accordingly, various embodiments of the face shield 10 are low cost and disposable.

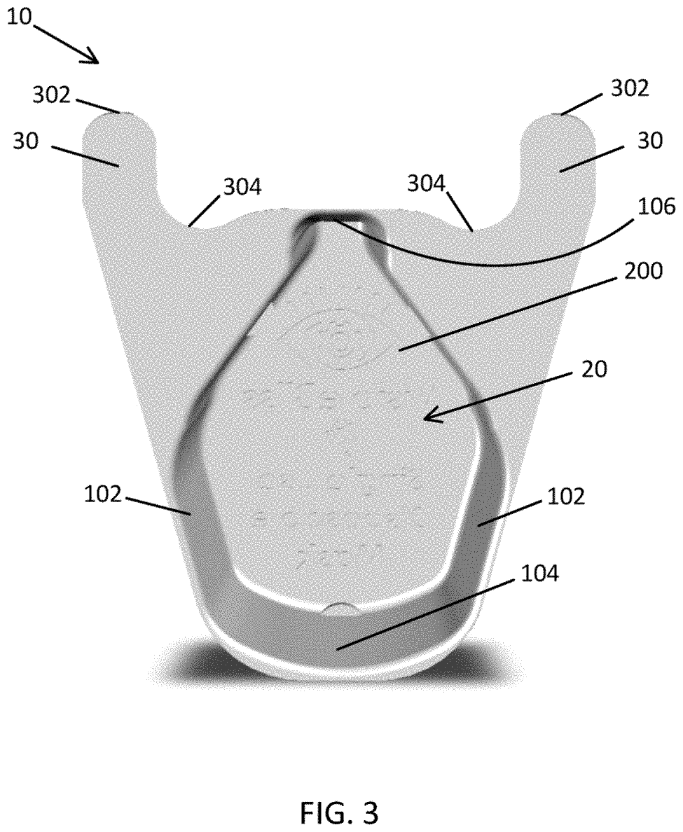

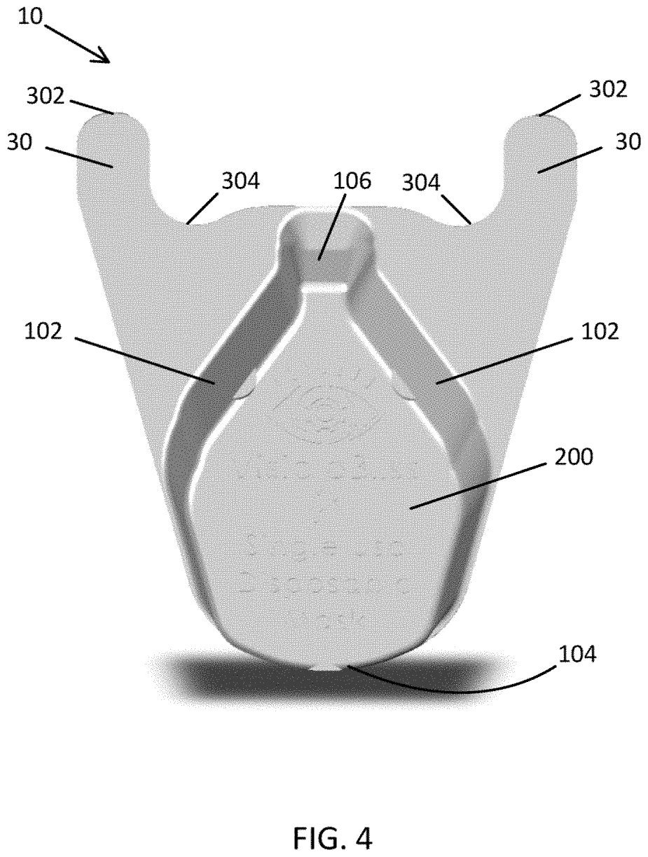

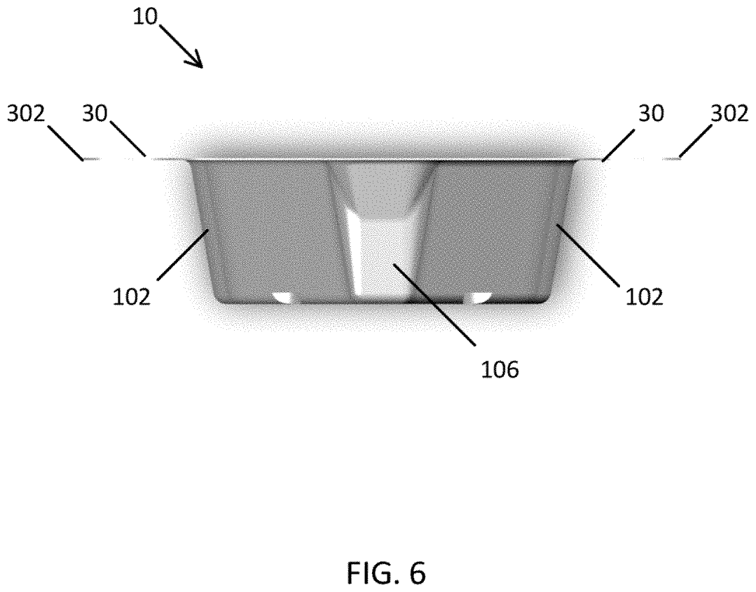

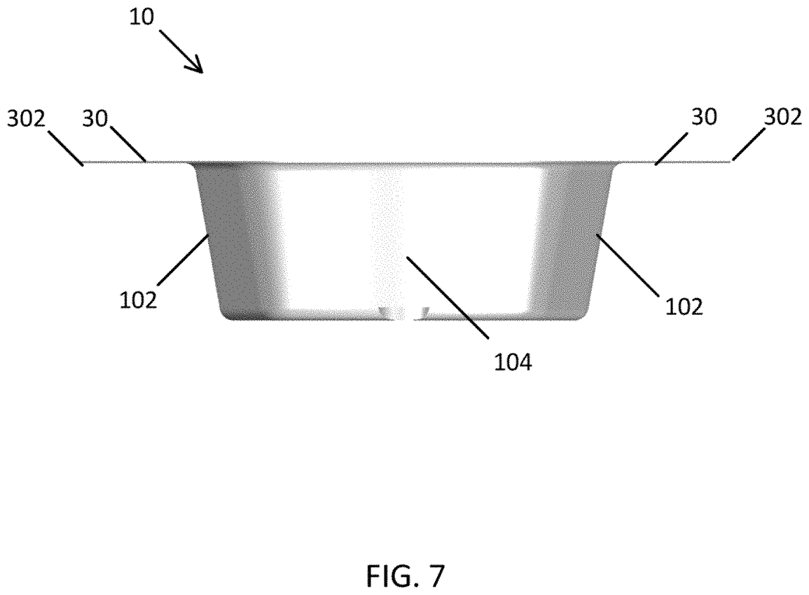

FIGS. 3-7 show additional views of the face shield 10. In the illustrated embodiment, the central cavity's sidewalls 100 comprise lateral sidewalls 102, a lower sidewall 104, and an upper sidewall 106. As shown in FIGS. 3 and 4, for example, the lateral sidewalls 102 define the lateral sides of the central cavity 20 and extend outwardly from the lateral perimeter of the front wall 200. The lower sidewall 104 and upper sidewall 106 extend between the lateral sidewalls 102 at lower and upper portions of the central cavity 20, respectively.

As is also shown in FIGS. 3-7, the face shield's side tabs 30 comprise flanges having a shape defined their outer perimeter. In the illustrated embodiment, the side tabs 30 extend outwardly and upwardly from the upper lateral sides of the central cavity 20. In particular, the uppermost portions 302 of the side tabs 30 (i.e., tips) are positioned above the central cavity 20.

The outer perimeter of the side tabs 30 also define recessed areas 304 positioned on lateral sides of the central cavity's upper sidewall 106. The recessed areas 304 are generally dimensioned for alignment with the orbital region of an adult person's face. In the illustrated embodiment, the recessed areas 304 are concave. As such, the recessed areas 304 ensure that—when the face shield 10 is attached to a phoropter—the face shield 10 does not obstruct the patient's eyes.

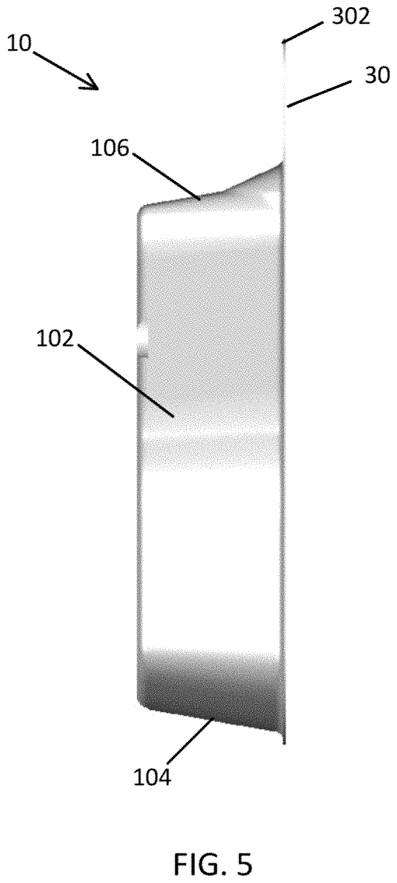

As can be appreciated from FIGS. 5-7, the side tabs 30 are thin and substantially flat. Additionally, the side tabs 30 are positioned in a plane substantially parallel to the plane of the opening of the central cavity 20. For example, the side tabs 30 are positioned in the same plane as the opening of the central cavity 20. In other words, the side tabs 30 are oriented substantially perpendicular to the sidewalls 100 and positioned immediately adjacent the perimeter edges of the sidewalls 100 on the patient-side of the face shield 10.

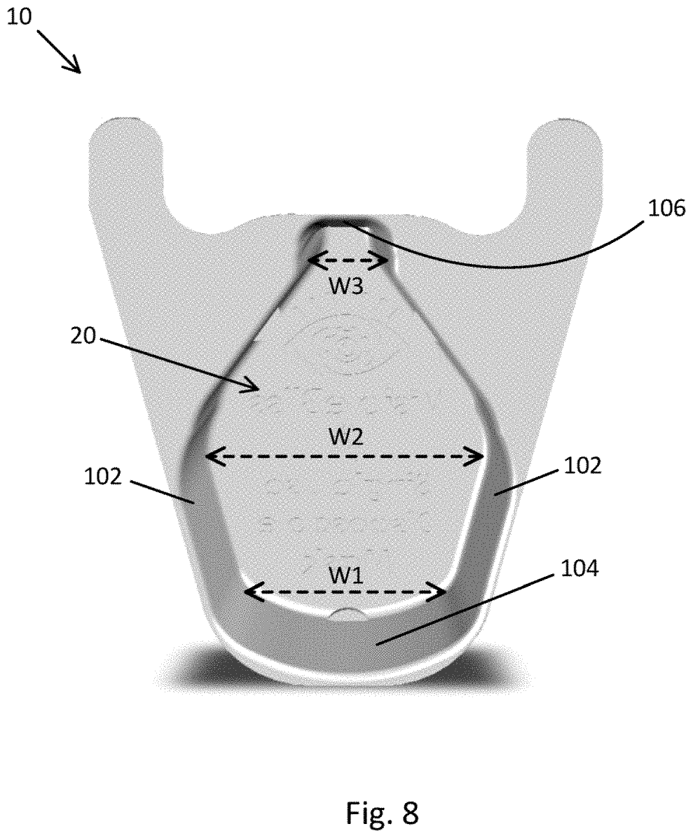

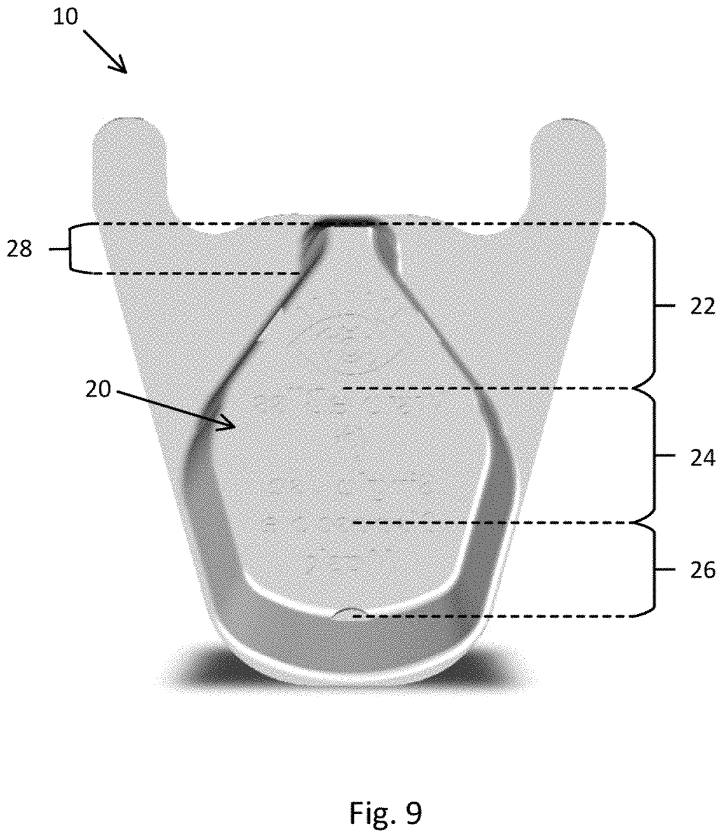

As shown in FIGS. 8 and 9, the lateral sidewalls 102 are spaced apart from one another to define the width of the central cavity 20. In particular, the lateral sidewalls 102 are shaped such that the width of the central cavity 20 varies at different locations. For example, as shown in FIG. 8, a bottom portion of the central cavity 20 has a width W1 (e.g., the distance between the opposing lateral sidewalls 102 adjacent the lower sidewall 104). In various embodiments, the width W1 is generally dimensioned so as to span the mental region 2006 of an adult person's face. Accordingly, as shown in FIG. 9, the central cavity 20 defines a lower cavity region 26 dimensioned to accommodate the mental region 2006 of an adult person's face.

As further illustrated in FIG. 8, a medial portion of the central cavity 20 has a width W2 (e.g., the distance between the opposing lateral sidewalls 102 proximate to a middle portion of the central cavity 20). In various embodiments, the width W2 is generally dimensioned so as to span the oral region 2004 of an adult person's face. As can be appreciated from FIG. 8, the width W2 is larger than the width W1, making the central cavity 20 narrower at its bottom portion (proximate to the lower sidewall 104) than at the middle portion. As shown in FIG. 9, the central cavity 20 defines a middle cavity region 24 dimensioned to accommodate the oral region 2004 of an adult person's face. As can be appreciated from FIG. 9, the middle cavity region 24 is generally wider than the lower cavity region 26.

Referring back to FIG. 8, an upper portion of the central cavity 20 has a width W3 (e.g., the distance between the opposing lateral sidewalls 102 proximate to the upper sidewall 106). In various embodiments, the width W3 is generally dimensioned so as to span the bridge of an adult person's nose, while the width of the central cavity 20 between the widths W2 and W3 is dimensioned to span the nasal region 2002 of an adult person's face. Accordingly, as shown in FIG. 9, the central cavity 20 defines an upper cavity region 22 dimensioned to accommodate the nasal region 2002 of an adult person's face. As further illustrated in FIG. 9, the central cavity's upper cavity region 22 includes a bridge region 28, which is dimensioned to accommodate the bridge of an adult person's nose.

As illustrated in FIG. 8, the width W3 is narrower than (e.g., less than) the width W2 and narrower than the width W1. Accordingly, as shown in FIGS. 8 and 9, the central cavity's upper cavity region 22 is generally narrower than the central cavity's middle cavity region 24 and lower cavity region 26 (the lower cavity region 26 being generally narrower than the middle cavity region 24).

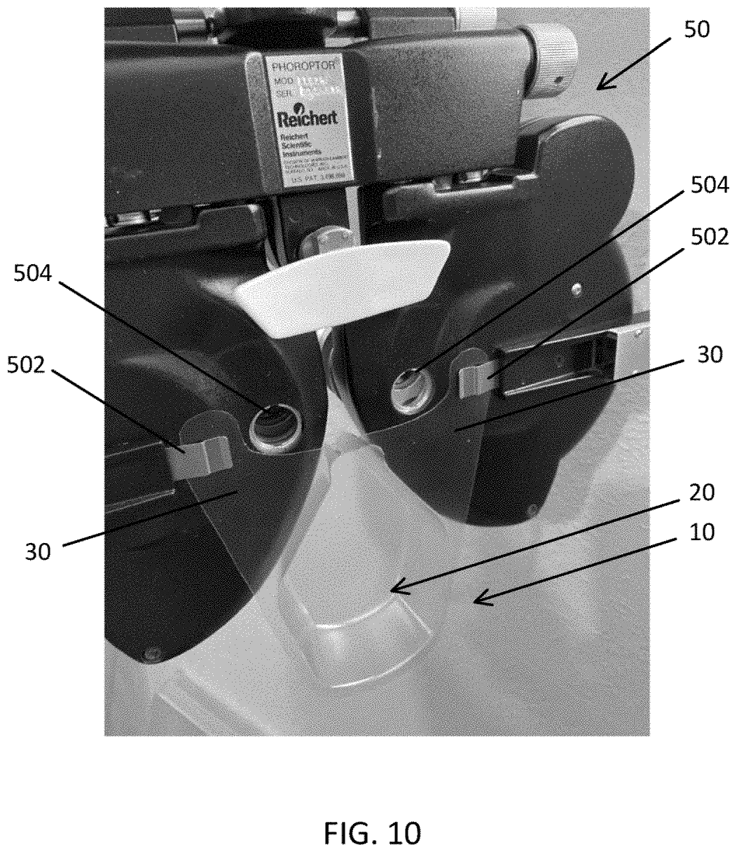

FIG. 10 shows the face shield 10 secured to a manual phoropter 50. In the illustrated embodiment of FIG. 10, the phoropter 50 includes clips 502 (e.g., cheek guard clips) and lens housings 504 (e.g., through which a patient looks through during an optical examination). The phoropter's clips 502 are made of a resilient metal (e.g., stainless steel) biased towards the surface of the phoropter. Accordingly, as shown in FIG. 10, the face shield 10 can be positioned such that its side tabs 30 are inserted between the clips 502 and the surface of the phoropter 50. In this way, the side tabs 30 enable the face shield 10 to be secured in a fixed position relative to the phoropter 50 by the clips 502.

As can be appreciated from FIG. 10, when the face shield 10 is secured to the phoropter 50 via the clips 502, the central cavity 20 is positioned between the phoropters lens housings 504. Accordingly, when the patient's eyes are aligned with the phoropter's lens housings 504, the face shield's central cavity 20 is positioned to shield at least the nasal, oral, and mental regions of the patient's face. As a result, the impermeable face shield 10 redirects breath from a patient's nose and/or mouth (e.g., by capturing breath from a patient in the central cavity 20 and/or blocking and reversing the flow of breath back towards the patient). In this way, the face shield 10 prevents the patient's breath from flowing toward the optometrist on the opposite side of the phoropter. In an embodiment, the face shield 10 redirects the flow of breath through the lower region of the central cavity 20. Furthermore, when the face shield 10 is secured to the phoropter 50, the recessed areas 304 of the side tabs' outer perimeter are configured for being positioned beneath and aligned with the phoropter's lens housings 504. This configuration ensures the face shield 10 does not obstruct the patient's view into the phoropter's lens housings 504.

As will be appreciated from the description herein, various aspects of the face shield 10 may be modified in alternative embodiments. For example, in certain embodiments, the central cavity 20 may be provided without a lower sidewall 104. In such embodiments, the lower portion of the central cavity 20 may be open (with the remaining lateral sidewalls 102 and upper sidewall 106 surrounding the patient's face). In other embodiments, the sidewalls 100 may define a substantially ovular shape dimensioned to mask a patient's face. Although the face shield 10 illustrated herein is configured for covering the nasal, oral, and mental regions of a patient's face, in various other embodiments the face shield 10 may include wider flanges and other areas to shield additional portions of a user's face.

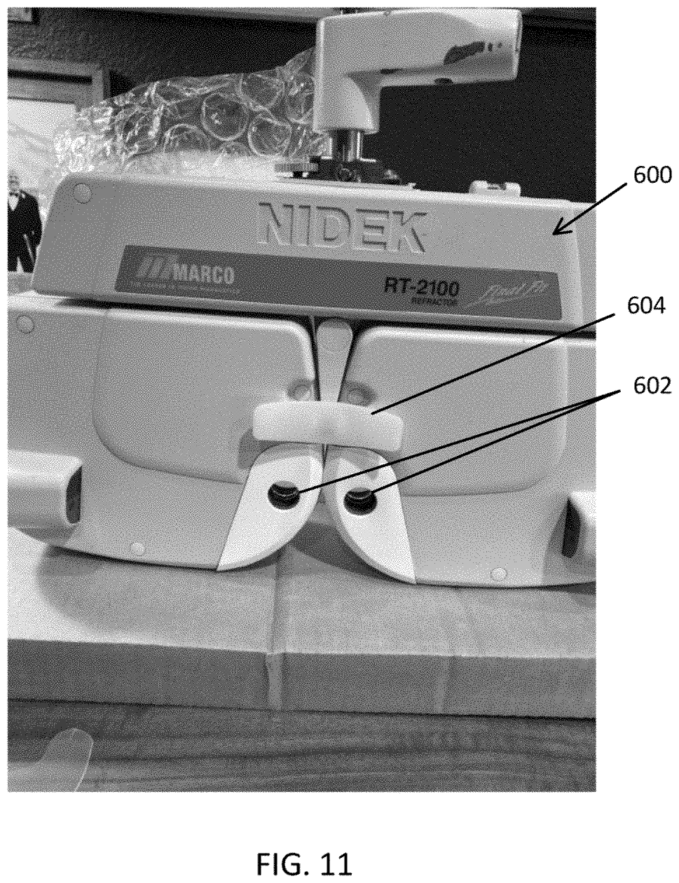

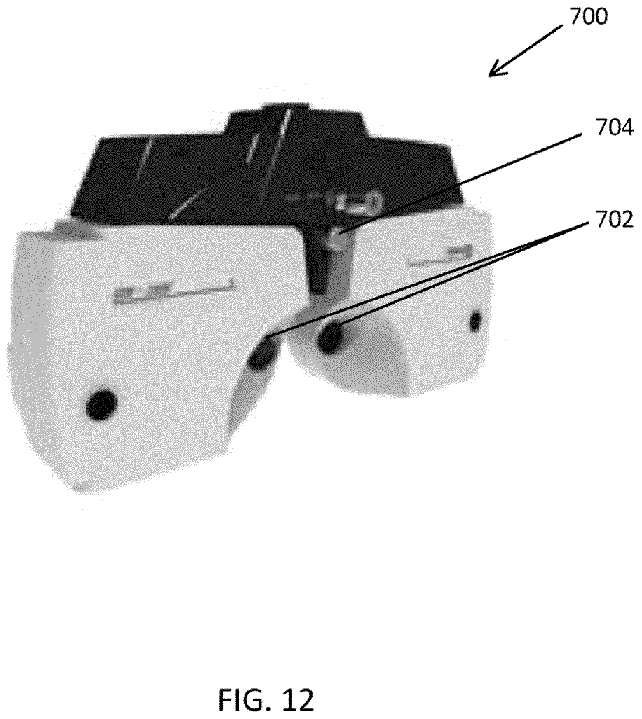

As noted earlier herein, certain phoropters are not equipped with cheek guard clips (e.g., the clips 502 shown in FIG. 10). However, phoropters lacking these clips (typically automated phoropters) often include an outwardly protruding feature above the phoropter's lenses. For example, FIG. 11 illustrates an automated phoropter 600 having lens housings 602 and a forehead rest 604. FIG. 12 shows another phoropter 700 having lens housings 702 and an outwardly protruding knob 704.

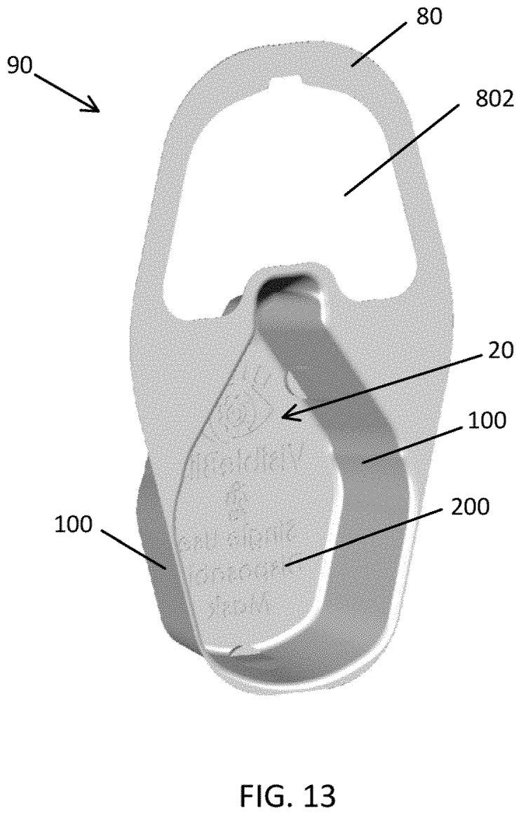

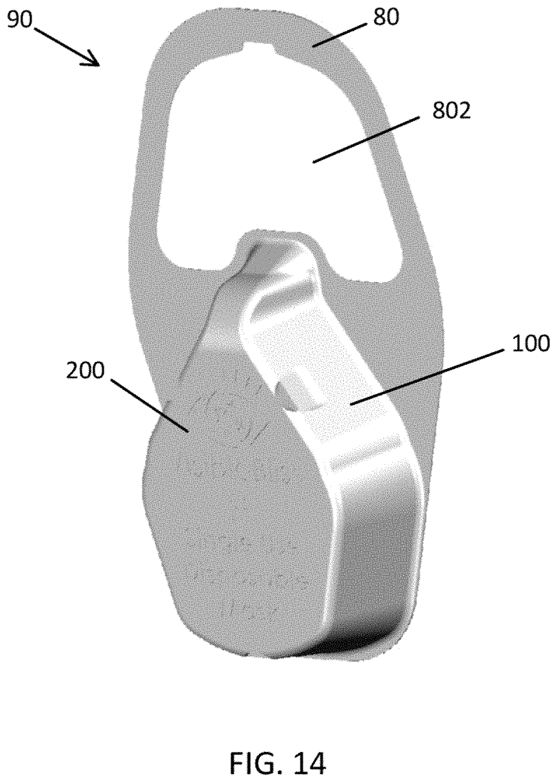

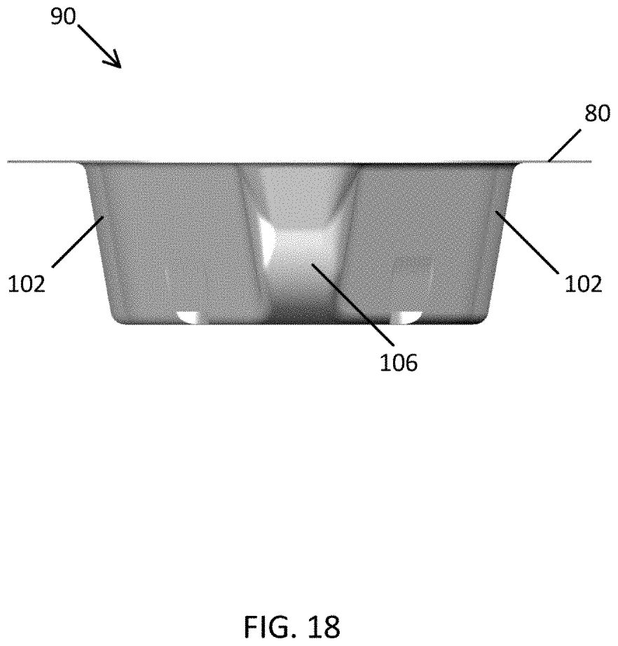

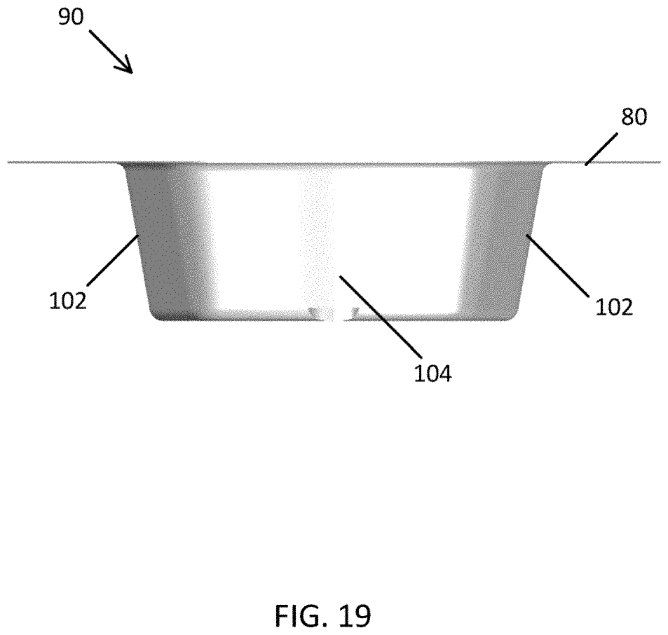

FIGS. 13-19 show an alternative embodiment of the face shield 10 in which the side tabs 30 have been replaced by an upper flange 80 defining a central aperture 802 (the face shield shown in FIGS. 13-19 will be referred to herein as the face shield 90 for clarity). In the illustrated embodiment of FIGS. 13-19, the central cavity 20 of the face shield 90 is substantially identical to the central cavity 20 of the face shield 10 shown in FIG. 1 (e.g., the sidewalls 100 and front wall 200 are the same). However, in the embodiment of FIGS. 13-19, the face shield 90 includes an upper flange 80 which extends upwardly and outwardly from lateral portions of the face shield 90. In particular, the upper flange 80 defines a central aperture 802 located above the central cavity 20. As discussed in greater detail herein, the upper flange's central aperture 802 is configured to permit the face shield 90 to be hung from an outwardly protruding feature on the patient-side of a phoropter (e.g., the forehead rest 604 shown in FIG. 11 or the knob 704 shown in FIG. 12). The upper flange 80 therefore functions as a loop or halter to facilitate hanging of the face shield 90. In this way, the upper flange 80 is an attachment feature of the face shield 90.

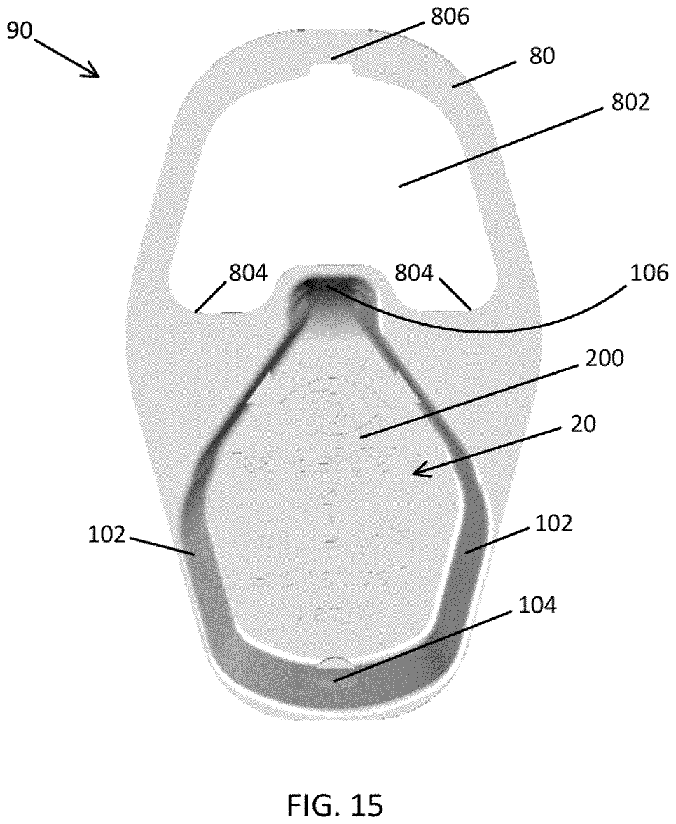

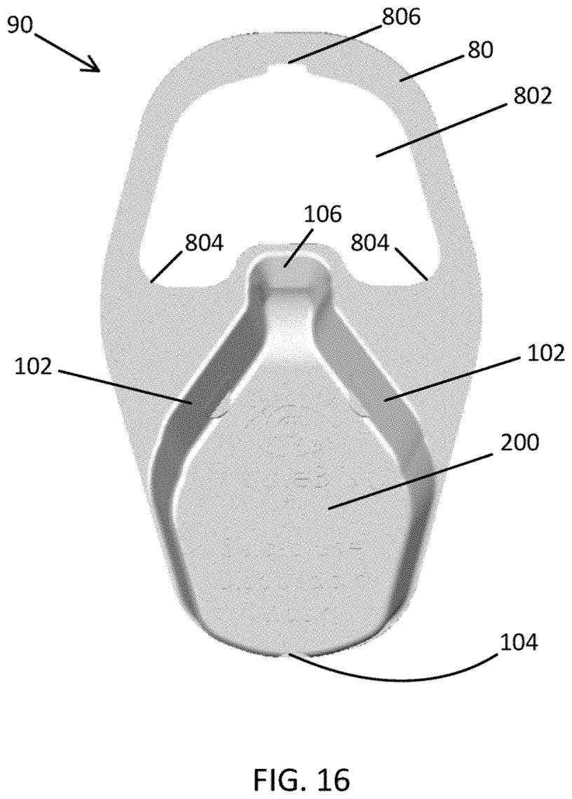

As shown in FIGS. 15 and 16, the inner perimeter of the upper flange 80—which defines the central aperture 802—includes recessed areas 804 positioned on lateral sides of the central cavity's upper sidewall 106. In the illustrated embodiment, the recessed areas 804 are generally concave and dimensioned for alignment with the orbital region of an adult person's face. Accordingly, the recessed areas 804 ensure that—when the face shield 90 is hung from an outwardly protruding portion of the phoropter—the face shield 90 does not obstruct the patient's eyes.

In addition, the inner perimeter of the upper flange 80 defines a notch 806. In various embodiments, the notch 806 is configured engage an outwardly protruding portion of the phoropter (e.g., the forehead rest 604 in FIG. 11 or the knob 704 in FIG. 12). As shown in FIGS. 15, and 16, the notch 806 is generally aligned above the upper sidewall 106 of the face shield's central cavity 20. Accordingly, when the face shield 90 is hanging on a phoropter from the flange 80, the notch helps retain the positioning of the face shield's central cavity 20 between the phoropter's lens housings (e.g., the lens housings 602 in FIG. 11 or the lens housings 702 in FIG. 12).

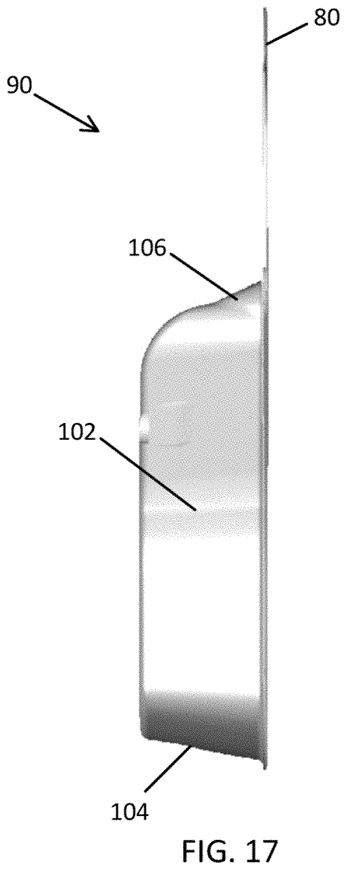

As can be appreciated from FIGS. 17-19, the upper flange 80 is thin and substantially flat. Additionally, the upper flange 80 is positioned in the same plane as the opening of the central cavity 20. In other words, the upper flange 80 is oriented substantially perpendicular to the sidewalls 100 and is positioned immediately adjacent the perimeter edges of the sidewalls 100 on the patient-side of the face shield 90.

As with the face shield 10 shown in FIGS. 1-9, the face shield 90 of FIGS. 13-19 is formed from a single piece of material. For example, in the illustrated embodiment of FIGS. 13-19, the face shield 90 is formed from a thin plastic sheet molded to define the central cavity 20 and upper flange 80. Plastics suitable for forming the face shield 90 include, but are not limited to, polyethylene, polypropylene, polyethylene terephthalate, acrylics, and the like. Accordingly, various embodiments of the face shield 90 are low cost and disposable.

While various embodiments have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above. Accordingly, the specification is intended to embrace all such modifications and variations of the disclosed embodiments that fall within the spirit and scope of the appended claims.

In addition, various inventive concepts may be embodied as one or more methods, of which an example has been provided. The acts performed as part of the method may be ordered in any suitable way. Accordingly, embodiments may be constructed in which acts are performed in an order different from illustrated, which may include performing some acts simultaneously, even though shown as sequential acts in illustrative embodiments.

1. A face shield for use with a phoropter, the face shield comprising:
a central cavity defined by a front wall and one or more sidewalls extending outwardly from a perimeter of the front wall, wherein the central cavity is formed from an impermeable material and is configured for surrounding a nasal region, an oral region, and a mental region of an adult person's face; and
one or more attachment features extending outwardly from the central cavity and configured for attaching the face shield to the phoropter.

2. The face shield of claim 1, wherein the one or more sidewalls comprise two lateral sidewalls, a bottom sidewall, and a top sidewall.

3. The face shield of claim 1, wherein the central cavity defines a plurality of cavity regions comprising an upper region, a middle region, and a lower region;
wherein the central cavity's upper region defines a width configured to accommodate the nasal region of the adult person's face;
wherein the central cavity's middle region defines a width configured to accommodate the oral region of the adult person's face; and
wherein the central cavity's lower region defines a width configured to accommodate the mental region of the adult person's face.

4. The face shield of claim 3, wherein the width defined by the central cavity's upper region is less than the width defined by the central cavity's middle region and less than the width defined by the central cavity's lower region.

5. The face shield of claim 3, wherein the width of the central cavity's upper region is further configured to be positioned between lens housings of the phoropter.

6. The face shield of claim 1, wherein the central cavity defines a cavity opening, and the one or more attachment features extend outwardly from the one or more sidewalls in a plane substantially parallel to a plane of the cavity opening.

7. The face shield of claim 1, wherein the one or more attachment features comprise a first side tab extending outwardly and upwardly from a first side of the central cavity, and a second side tab extending outwardly and upwardly from a second side of the central cavity.

8. The face shield of claim 7, wherein each of the first side tab and the second side tab are configured to be secured to a surface of a patient-side of the phoropter by preexisting cheek guard clips.

9. The face shield of claim 1, wherein the one or more attachment features comprise an upper flange extending upwardly from the central cavity and defining an aperture above the central cavity.

10. The face shield of claim 9, wherein an inner perimeter of the upper flange's aperture defines a notch configured to be positioned on an outwardly protruding member of the phoropter.

11. The face shield of claim 1, wherein the face shield is formed from a single piece of impermeable material selected from a group consisting of: polyethylene, polypropylene, polyethylene terephthalate, and acrylics.

12. A method for securing a face shield to a patient-side of a phoropter for protection of a patient, the face shield comprising a central cavity defined by a front wall and one or more sidewalls, wherein the face shield's central cavity defines an upper cavity region, a middle cavity region, and a lower cavity region, and wherein the face shield further comprises one or more attachment features extending outwardly from the central cavity, the method comprising:
positioning the upper cavity region of the face shield between lens housings of the phoropter; and
attaching the one or more attachment features of the face shield to one or more securing components of the patient-side of the phoropter.

13. The method of claim 12, wherein the face shield's one or more attachment features comprise two side tabs, and the one or more securing components of the phoropter comprise two preexisting cheek guard clips attached to and biased towards a surface of the patient-side of the phoropter; and
wherein the step of attaching the one or more attachment features of the face shield to one or more securing components of the phoropter comprises securing each of the face shield's side tabs between one of the preexisting cheek guard clips and the surface of the phoropter.

14. The method of claim 12, wherein the face shield's one or more attachment features comprise an upper flange defining an aperture above the central cavity, and the one or more securing components of the phoropter comprise an outwardly protruding member; and
wherein the step of attaching the one or more attachment features of the face shield to one or more securing components of the phoropter comprises positioning the face shield's upper flange on the outwardly protruding member such that the outwardly protruding member is positioned within the upper flange's aperture.
